(19) United States
(12) Patent Application Publication
(10) Pub. No.: US 2022/0362534 A1
(43) Pub. Date: Nov. 17, 2022

(54) SPHERULE INSERTION TOOLS

(71) Applicant: David S. Goldsmith, Atlanta, GA (US)

(72) Inventor: David S. Goldsmith, Atlanta, GA (US)

(21) Appl. No.: 17/865,025

(22) Filed: Jul. 14, 2022

Related U.S. Application Data
(63) Continuation-in-part of application No. 15/932,172, filed on Feb. 14, 2018, now Pat. No. 11,389,171.
Application No. 15/932,172 is a continuation-in-part of application No. 13/694,835, filed on Jan. 9, 2013, now abandoned.
Application No. 13/694,835 is a continuation-in-part of application No. 11/986,021, filed on Nov. 19, 2007, now abandoned.
(60) Provisional application No. 60/860,392, filed on Nov. 21, 2006.

(51) Int. Cl. A61M 37/00; A61K 9/16

(52) U.S. Cl. CPC A61M 37/0069; A61K 9/16; A61M 2205/07; A61M 2205/0272; A61B 18/082

(57) ABSTRACT

Described are surgical tools to facilitate the proper implantation beneath the outer layer of tubular anatomical structures, or ductus, to include vessels, the trachea, esophagus, gut, and ureters, as well as the outer layer or within the parenchyma of organs, glands, or other tissue, of medicinal, magnetically susceptible, magnetized, and/or radiation-emitting spherules sized in proportion to the substrate structure. Spherule insertion tools expedite insertion transluminally to implant the wall surrounding a lumen making possible therapy and/or extraluminal stenting which leaves the lumen clear for subsequent passage. Spherules can also be introduced into deeper tissue through a ‘keyhole’ incision at the body surface. For evolving methods calling for the placement of numerous ‘seeds’ and/or boluses, eliminated are the need for more extensive incision with increased trauma, procedural duration, and healing time. Avoidance of the lumen is augmented by placing the magnets subcutaneously rather than using a magnetized perivascular collar, or stent-jacket.

### RELATED U.S. APPLICATION DATA

This application is a continuation-in-part of U.S. application Ser. No. 15/932,172, filed on Feb. 14, 2018, which is a continuation-in-part of U.S. application Ser. No. 13/694,835, filed on Jan. 9, 2013, which is a continuation-in-part of U.S. application Ser. No. 11/986,021, filed on Nov. 19, 2007, which claims the benefit of U.S. Provisional Application No. 60/860,392, filed on Nov. 21, 2006, applications which are incorporated herein by reference in their entireties.

### BACKGROUND OF THE INVENTION

### 1. Field of the Invention

This divisional application addresses tools for the insertion of medicinal, radioactive, and/or magnetically retractable spherule implants, also referable to as spherule inserters or spherule injectors intended for use by veterinary specialists, pulmonologists, gastroenterologists, urologists, endourologists, nephrologists, hepatologists, interventional radiologists, and cardiologists, internists, gynecologists, and general, endocrine, oncological, cardiovascular, cardiothoracic, pediatric cardiac, and neurosurgeons to infix small spheroidal implants, or spherules (minispherules, microspherules, or more often, miniballs) beneath the outer or adventitial layer or tunic of tubular anatomical structures or more deeply within the parenchyma of any anatomical structure, to include an organ, gland, lymph node, or a volume of tissue.

### 2. Miniballs

Small spheroidal implants, miniballs typically consist of medicinal, such as anti-inflammatory, antimicrobial, chemotherapeutic, or anesthetizing substances, sealed irradiating ‘seeds,’ or ‘smart pill’ sensors, or these in a variety of combinations, usually containing sufficient magnetically susceptible soft iron-silicon to make possible the extraluminal stenting of the substrate ductus under a surrounding magnetic pull, or when necessary or desired, in a lesser amount to allow the quick recoverability of a mispositioned or errant implant.

Just as radioactive seeds are currently used, miniballs can also be used as fiducial marker guides for resection, radiation, or cyberknife stereotactic radiosurgery. In that neither can be favorably substituted with a type implant of alternative form factor or means for insertion into tissue, miniballs and the tools used to insert these relate to one another in an exclusive manner and are thus properly viewed as obligatory constituents comprising a single invention.

Whereas an aeroballistically inserted brachytherapeutic spherule implant is placed beside or inside a neoplasm to be eradicated, an implant containing ferrous metal, positioned inside the neoplasm, whether also a sealed spheroidal source of radiation otherwise the same as a permanent prostate seed, can also destroy malignant tissue through the cytotoxicity of heat when an implant with ferrous content is placed in a high frequency alternating magnetic field (see, for example, Arduino, A., Zanovello, U., Hand, J., Zilberti, L., Bruhl, R., Chiampi, M., and Bottauscio, O. 2021. “Heating of Hip Joint Implants in MRI: The Combined Effect of RF and Switched-gradient Fields,” Magnetic Resonance in Medicine 85(6):3447-3462; Chandrasekharan, P., Tay, Z. W., Hensley, D., Zhou, X. Y., Fung, B. K. L., and 11 others 2020. “Using Magnetic Particle Imaging Systems to Localize an Guide Magnetic Hyperthermia Treatment: Tracers, Hardware, and Future Medical Applications,” Theranostics 10(7):2965-2981; Liu. X., Zhang, Y., Wang, Y., Zhu, W., Li, G., and 9 others 2020. “Comprehensive Understanding of Magnetic Hyperthermia for Improving Antitumor Therapeutic Efficacy,” Theranostics 10(8):3793-3815; Chopra, R., Shaikh, S., Chatzinoff, Y., Munaweera, I., and 6 others 2017. “Employing High-frequency Alternative Magnetic Fields for the Non-invasive Treatment of Prosthetic Joint Infections,” Scientific Reports 7:7520; Matsui, H., Hamuro, M., Nakamura, K., Kayahara, H., Murano, K., Kotsuka, Y., and Miki, Y. 2012. “Development of a Highly Efficient Implanted Thermal Ablation Device: In vivo Experiment in Rat Liver,” British Journal of Radiology 85(1017):e734-e739). Moreover, “Sublethal heat doses sensitize cancer cells to radiation and drugs.” (DeNardo, G. L., and DeNardo, S. J. 2008. “Update: Turning the Heat on Cancer,” Cancer Biotherapy and Radiopharmaceuticals 23(6):671-679).

Therapeutic stays, small generally arcuate bands or tangs placed with a stay insertion tool, addressed in a copending application entitled Stay Insertion Tools, and spherules are both small implants with a shape or form factor that allows these to be positioned within the wall surrounding a tubular anatomical structure, or ductus. Conventional means for the insertion of radioactive prostate seeds, for example, are incapable of positioning implants thus. The hollow needles used insert implants rectilinearly despite the fact that many ductus walls lack the thickness to allow insertion thus so that the adaxial end of the implant is brought too close if not penetrates into the lumen. Rather, positioning implants ductus-intramurally requires means for insertion that allow infixion in concentric relation to the wall of the ductus.

Insertion about the circumference of the wall makes possible the dilatation of the ductus under the pull of a surrounding magnetic field as a form of periductal, hence extraluminal, stenting, or the infixion of therapeutic implants whether medicinal, radioactive, chemotherapeutic, or these in various combinations, or the placement of radioactive implants to serve as guides for the use of stereotactic apparatus. Primarily intended for implanting the walls of ductus, stay and spheroid implant insertion tools are not intended to replace but rather to supplement conventional means for implantation where the angle of approach or the location of both ductal and nonductal structures make the use of conventional means such a seed insertion needles if not impossible, then awkward and time consuming.

For example, in metastatic cancer where shed or ‘daughter cells’ of the primary or ‘mother’ tumor have been dispersed to and have taken hold in multiple sites throughout the body, the positioning of irradiating implants near each malignant lesion as brachytherapeutic can materially aid patients unable to withstand open surgery. Such a supplementation of the abscopal effect as addressed below with direct treatment of the metastases would reasonably be expected to elicit a significant effect. The object then is to achieve if not a cure, then at least a significant palliative relief of symptoms. The concept is that to make possible the infixion of irradiating as well as adjuvant implants into tissue at different angles and into different types of anatomical structures through a small incision through the integument, the scrub nurse will have assembled a set of prostate seed insertion needles and stay and spherule insertion tools, as well as ordinary hypodermic needles, to allow their use interchangeably.

In the roof of the trachea, periductal stenting by means of magnetic retraction can be used to alleviate the suffocation of a small dog with a collapsed trachea, for example, and in the wall surrounding the lumen of a tubular anatomical structure to include blood vessels, this extraluminal form of stenting leaves the lumen clear for the later passage therethrough of a transluminal or miniature cabled device such as an angioscope, intravascular ultrasound probe, or laser, where the endothelium is left untouched so that the risk of restenosis as sometimes occurs with an endoluminal stent has been eliminated. Stenting in this manner is accomplished perivascularly by surrounding the ductus with a collar magnetized perpendicularly to the axis of the ductus, or when practicable to reduce the degree of invasiveness, by small but powerful neodymium iron boron permanent magnets positioned subcutaneously.

The copending application entitled Stay Insertion Tools describes an alternative form of small implant—stays, which are small, usually arcuate, band or tang-shaped implants for insertion into substrate tissue from outside the surface of that tissue thus avoiding the lumen of a ductus not just following, as is true with miniballs, but even during their placement. Spherules, or miniballs, are placed transluminally, or transluminally, although as indicated for stays, once placed these too leave the lumen clear for follow up transluminal procedures. Once introduced, spontaneous closure by the tissue of the small breach along the trajectory due to swelling behind the implant prevents its release, inflammation consequent to the insertion of miniballs lessened by coating each with an anti-inflammatory such as prednisolone or dexamethasone, which both stay and spherule insertion tools can accomplish automatically.

The absolute amount of steroids involved is far too small to provoke any of the serious side effects associated with these drugs. Nevertheless, whereas a transluminal procedure can be performed immediately following the insertion of stays, which may cause some minor swelling but not perforation into the lumen, following the insertion of miniballs, a brief period for healing should be allowed. Whereas stays necessitate access through a small incision at the body surface, a transluminal spheroid delivery miniball insertion tool, or barrel-assembly, avoids this through insertion into the body no differently than would a conventional angioplasty catheter, typically through a small Seldinger cut-down incision most often into a femoral or radial artery, but without the need for, and therefore without the risks associated with, the use of a guidewire.

The external approach afforded by stays can make it possible to implant the wall of a ductus which malacotic, diseased, or otherwise susceptible to incisions, abrasions, or even perforations must not be implanted transluminally as are spherules from within the lumen. Whereas stays are generally positioned to a variable depth closer to the surface of the substrate anatomical structure, each successive transluminally discharged spheroidal implant can generally be inserted to a greater depth. Spheroidal implants in a train, for example, can be made to progressively descend, ascend, or alternate in depth as optimal for the conformation of the structure implanted. This adjustability is made possible by the multiple means provided for adjusting the discharge or exit velocity incorporated into both the interventional airgun such as that shown here in FIG. 8 and FIGS. 81 and 82 of the parent application Ser. No. 15/932,172, as well as in the airgun hand piece.

Aeroballistic insertion into the wall of a vessel based upon actual pretesting of the tissue with a device which allows adjustment in the discharge, or exit velocity should seldom if ever result in an errant spheroid that would enter into the lumen, which in a vessel would represent the entry of an embolism into the circulation. However, multiple means are provided for preventing embolization. These include a run-ahead embolic filter, recovery magnets in the head of the barrel-assembly, a powerful upstream interdiction magnet, and the containment of a discharged miniball inside the margin surrounding the discharge holes in the barrel assembly muzzle head.

Miniball insertion tools of the pistol type shown in FIG. 1 also incorporate magnets to retrieve an errant miniball despite the fact that this type of insertion device is meant for larger nonvascular ductus such as the trachea or for use in an open surgical field. Depending upon accessibility and risk avoidance, the clinician can alternate between the various types of medicinal, radioactive, and/or tractive implants and the tools used to place these during a single procedure. Miniball discharge devices are shown in FIGS. 1 and 2, which are the same as FIGS. 32 and 65 in the parent application Ser. No. 15/932,172.

FIG. 1 shows a pistol-configured device for use as indicated in an open surgical field or a lumen adequately larger in diameter than the discharge end of the device to allow its free movement. FIG. 2 shows a transluminal barrel-assembly type spherule insertion tool best used in a lumen equal in diameter to its muzzle head when the escape of a miniball into the lumen when in flush contact with the endothelium or urothelium round and about is prevented by enclosure within the margin encircling the exit holes.

In a narrower lumen, it is therefore best to use a barrel-assembly with a muzzle head that matches the caliber of the lumen to be implanted. Then both sides of the barrel-assembly can be used. If a barrel-assembly of smaller diameter than the lumen is used, only the one exit hole can be kept flush to the endothelium. Use thus requires that both the miniball holding rotary clip and the muzzle-head be clearly marked with contrast to ensure that loading and discharge will be limited to the side intended for use.

Stays and spherules make possible the highly localized insertion of implants which can incorporate different formulations of component drugs, for example, with or without magnetically susceptible or contained radioactive content. The substances contained can be concentrically layered where the layers are formulated for consecutive release, or can be coated so that when the outer envelope is absorbed, there are released smaller implants which can in turn be layered or consist of microparticles or even in situ diagnostic or analytic Dynabeads® (Dyno Industrier, Lier, Norway), for example, selective for certain targets before extraction for analysis (see, for example, Fujiwara, Y., Tanno Y., Sugishita, H. Kishi, Y. Makino, Y. and Okada, Y. 2021. “Preparation of Optimized Concanavalin A-conjugated Dynabeads® Magnetic Beads for CUT&Tag [cleavage under targets and tagmentation],” PLoS [Public Library of Science] 16(11):e0259846; Sall, A., Corbee, D., Vikstrom, S., Ottosson, F. Persson, H., and Waldemarson, S. 2018. “Advancing the Immunoaffinity Platform AFFIRM [affinity selected reaction monitoring] to Targeted Measurement of Proteins in Serum in the Pg/Ml [picogram per milliliter] Range,” PLoS [Public Library of Science] 13(2):e0189116; Sawaguchi, S. Ogawa, M., and Okajima, T. 2017. “Protocol for Notch-ligand Vinding Assays Using Dynabeads,” Bio Protocol 7(20):e2582).

These can be absorbable and drug-releasing, can include some which are magnetically susceptible, some which are sealed sources of radiation, and can release a variety of the same or different smaller implants. Where recovery may be necessary, only so much magnetically susceptible matter is incorporated as necessary, which will be less than when the implant is specifically intended for stenting. Thus, a miniball can be in the form of a solitary encapsulated continuous spherical bolus or a spherule containing one or more different substances distributed among smaller encapsulated or unencapsulated, absorbable or nonabsorbable, spherules within that larger, for example.

Miniballs can be layered with different drugs, and when each layer contains microspheres, the delay release time of each layer and even each type microsphere can be different to allow considerable extension of the duration of administration of the same or different drugs. The facility of placement is optimal when the condition treated necessitates an open surgical field anyway, giving relatively free access. Except for an almost invariably ineffectual trace amount carried to other parts of the body through the circulation, the insertion of a miniball into tissue targets the contents of the miniball to that specific site to the substantial exclusion of the rest of the body. Magnetic matter toxic, it is always isolated through encapsulation, making the inclusion of such matter necessarily central within its spherule.

Whereas magnetic flux does not affect drugs to any medically significant extent, ionizing radiation can significantly affect:

Drugs (see, for example, McGill, M. R., Findley, D. L., Mazur, A. Yee, E. U., Allard, F. D., and 5 others 2022. “Radiation Effects on Methamphetamine [in the past occasionally prescribed to suppress appetite as an aid to weight reduction, as a bronchial inhaler, nasal decongestant, or to treat attention deficit hyperactivity disorder] Pharmacokinetics and Pharmacodynamics in Rats,” European Journal of Drug Metabolism and Pharmacokinetics 47(3):319-330; Bocedi, A., Ingrosso, G. Cattani, G., Miceli, R., Ponti, E., and 6 others 2019. “The Impact of Ionizing Irradiation on Liver Detoxifying Enzymes. A Reinvestigation,” Cell Death Discovery 5:66; Li, X., Yan, J., Qiao, Y., Duan, Y., Xin, Y., Nian, Y., Zhu, L, and Liu, G. 2019. “Effects of Radiation on Drug Metabolism: A Review,” Current Drug Metabolism 20(5):350-360; Rendic, S. and Guengerich, F. P. 2012. “Summary of Information on the Effects of Ionizing and Non-ionizing Radiation on Cytochrome P450 and Other Drug Metabolizing Enzymes an Transporters” Current Drug Metabolism 13(6):787-814; Silinder, M. and Ozer, Y. 2012. “The Effect of Radiation on a Variety of Pharmaceuticals and Materials Containing Polymers,” Parenteral Drug Association Journal of Pharmaceutical Science and Technology 66(2): 184-199),

Stem cells, in addition to the damage done to all normal cells in its path (see, for example, Chapel, A. 2021. “Stem Cells and Irradiation,” Cells 10(4):760; Martinez, P. S., Giuranno, L., Vooijs, M. and Coppes, R. P. 2021. “The Radiation-induced Regenerative Response of Adult Tissue-specific Stem Cells: Models and Signaling Pathways,” Cancers 13(4):855; Platoff, R., Villalobos, M. A., Hagaman, A. R., Liu, Y., Matthews, M. and 3 others.2021. “Effects of Radiation and Chemotherapy on Adipose Stem Cells: Implications for Use in Far grating in Cancer Patients,” World Journal of Stem Cells 13(8):1084-1093; Squillaro, T., Galano, G., De Rosa, R. Peluso, G., and Galderisi, U. 2018. “Concise Review: The Effect of Low-dose Ionizing Radiation on Stem Cell Biology: A Contribution to Radiation Risk,” Stem Cells 36(8):1146-1153; Vallard, A., Espenel, S., Guy, J.-B., Diao, P., Xia, Y., and 6 others 2016. “Targeting Stem Cells by Radiation: From the Biological Angle to Clinical Aspects,” World Journal of Stem Cells 8(8):243-250; Mieloch, A. A. and Suchorska, W. M. 2015. “The Concept of Radiation-enhanced Stem Cell Differentiation,” Radiology and Oncology 49(3):209-216; Price, K. M. and Saran A. 2011. “Concise Review: Stem Cell Effects in Radiation Risk,” Stem Cells 29(9):1315-1321),

Inflammation, which radiation aggravates and augments (see, for example, Arnold, K. M., Opdenaker, L. M., Flynn, N. J., Appeah, D. K., and Sims-Mourtada, J. 2021. “Radiation Induces an Inflammatory Response that Results in STAT3 [signal transducer and activator of transcription 3]-dependent Changes in Cellular Plasticity and Radioresistance of Breast Cancer Stem-like Cells,” International Journal of Radiation Biology 96(4):434-447; Di Maggio, F., M., Minafra, L., Forte, G. I., Cammarata, F. P., Lio, D., and 3 others 2015. “Portrait of Inflammatory Response to Ionizing Radiation Treatment,” Journal of Inflammation (London, England) 12:14), making it clear that implants susceptible to significant degradative change if positioned within range of the radiation should not only be omitted from implants containing a source of radiation but kept beyond that range.

Radiation can interfere with an innate immune response but can also elicit an abscopal response which barring blockage by an immunosuppressive milieu inside the irradiated neoplasm vulnerable to immunomodulatory monoclonal antibody drugs such as ipilmumab and pembrolizumab, serve as adjuvant to stimulate the immune system to distribute the effect of the radiation, for example, to metastases remote from the target (see, for example, Dagoglu, N., Karaman, S., Caglar, H. B., and Oral, E. N. 2019. “Abscopal Effect of Radiotherapy in the Immunotherapy Era: Systematic Review of Reported Cases,” Cureus (Stanford, Calif.) 11(2): e4103; Kim, Y. J., Shin, H. J., Choi, M. E., Lee, W. J., Won, C. H., and 3 others 2019. “Radiation Dermatitis with Foreign Body Reaction Clinically Mimics a Cutaneous Metastasis from Breast Cancer,” Breast Journal 25(1):141-142; de Andrade Carvalho, H. and Villar, R. C. 2018. “Radiotherapy and Immune Response: the Systemic Effects of a Local Treatment,” Clinics (Sao Paulo, Brazil) 73(Supplement1):e5572; Brix, N., Tiefenthaller, A., Anders, H., Belka, C., and Lauber, K. 2017. “Abscopal, Immunological Effects of Radiotherapy: Narrowing the Gap between Clinical and Preclinical Experiences,” Immunological Reviews 280(1):249-279; Fend, L., Yamazaki, T., Remy, C., Fahrner, C., Gantzer, M. and 9 others 2017. “Immune Checkpoint Blockade, Immunogenic Chemotherapy or IFN-alpha [interferon alpha] Blockade Boost the Local and Abscopal Effects of Oncolytic Virotherapy,” Cancer Research 77(15):4146-4157; Rodriguez-Ruiz, M. E., Rodriguez, I., Garasa, S., Barbes, B., Solorzano, J. L., and 11 others 2016. “Abscopal Effects of Radiotherapy are Enhanced by Combined Immunostimulatory mAbs [monoclonal antibodies] and are dependent on CD8 T cells and Crosspriming,” Cancer Research 76(20):5994-6005; Victor, C. T.-S., Rech, A. J., Maity, A., Rengen, R., Pauken, K. E., and 15 others 2015. “Radiation and Dual Checkpoint Blockade Activate Non-redundant Immune Mechanisms in Cancer,” Nature 520(7547):373-377; Park, B., Yee, C. and Lee, K.-M. 2014. “The Effect of Radiation on the Immune Response to Cancers,” International Journal of Molecular Sciences 15(1):927-943).

Eliminating the need for invasive access to the target, the systemic administration of a drug, whether oral, intramuscular, or subcutaneous, for example, provides a distinct advantage. Unfortunately, it also imposes the need for a considerably increased dose to compensate for the dilution factor, and exposes the entire body to the drug. In the case of newly developed drugs, which are usually quite expensive, the need to increase the dose materially increases the cost for treatment, prompting the use instead of a less effective alternative.

Drug formulation can therefore discount the incorporation of adjuvant substances to target the drug on the basis of inherent affinity such as to incorporate iodine into a drug in order to obtain increased uptake by the thyroid gland, for example. In contrast, point of insertion application completely dispels this major drawback in the systemic dispersal of drugs which exposes all the tissues of the body to every drug posing numerous risks of complications. The drawback to point of insertion application being that it is necessarily invasive, the object is to reduce obtrusiveness of administration for both patient and provider to a minimum, and in this way, encourage the use of considerably more efficient medication.

Compared to injection, miniball insertion makes possible delivery in a form that 1. Can be fully sealed when a source of radiation, 2. Is contained as to allow a controllable rate of dispersal greater than might be attained by an increase in viscosity or a reduction in solubility, and 3. Is completely and immediately retrievable whether meant for temporary use, or later recovery if appropriate, 4. Is structurally unitary and of sufficient integrity to allow use when magnetically susceptible as the object subjected to traction for drawing tissue investing or substrate to it in the direction of attraction, making the dilatation of a stricture, for example, 5. Is compatible with the packaging of tiny sensors in the form of a spherules, and so that 6. When ejected aeroballistically, the implant can be delivered from a point not in immediate contact with the destination, affording a considerable advantage in positional flexibility.

Moreover, compared to a miniball, an injectant can incorporate time release minispherules to release different drugs, but as a whole, control over its dispersal is limited. Aeroballistic insertion might be characterized as analogous to jet injection for the insertion of a fluid medicinal where infixion is instead of a solid, albeit tiny, spherule implant of which the function can be not only medicinal as well as contained and therefore radioactive, as well as to serve in various mechanical capacities. That solid and contained implants are subject to migrate is shown in the case of permanent prostate brachytherapy seeds, which commonly are carried craniad through the venous circulation, such as via the periprostatic and hemorrhoidal venous plexuses (Nguyen, B. D. 2006. “Cardiac and Hepatic Seed Implant Embolization after Prostate Brachytherapy,” Urology 68(3):673.e17-e19).

When migration of a conventional radioactive prostate-type seed—which unlike stays and spherules are not immediately associated with means for their recovery—does occur, it is usually and innocuously to the lungs or liver, but rarely, to a kidney or a coronary artery with considerable nocuity (see, for example, Maletzki, P., Schwab, C., Markart, P., Engeler, D., Schiefer, J., Plasswilm, L., and Schmid, H. P. 2018. “Late Seed Migration after Prostate Brachytherapy with Iodine 125 Permanent Implants,” Prostate International 6(2):66-70; Sachdeva, S., Udechukwu, B. S., Elbelasi, H., Landwehr, K. P., St. Clair, W. H., and Winkler, M. A. 2017. “Prostate Brachytherapy Seed Migration to the Heart Seen on Cardiovascular Computed Tomographic Angiography,” Radiology Case Reports 12(1):31-33; Nakano, M., Yorozu, A. Saito, S., Sugawara, A., Maruo, S., and 5 others 2015. “Seed Migration after Transperineal Interstitial Prostate Brachytherapy by Using Loose Seeds: Japanese Prostate Cancer Outcome Study of Permanent Iodine-125 Seed Implantation Multi-institutional Cohort Study,” Radiation Oncology 10:228; Sugawara, A, Nakashima, J., Kunieda, E., Nagata, H., Mizuno, R., and 5 others 2011. “Incidence of Seed Migration to the Chest, Abdomen, and Pelvis after Transperineal Interstitial Prostate Brachytherapy with Loose Seeds,” Radiation Oncology 6, 130 (2011); Zhu, A. X., Wallner, K. E., Frivold, G. P., Ferry, D., Jutzy, K. R. and Foster, G. P. 2006. “Prostate Brachytherapy Seed Migration to the Right Coronary Artery Associated with an Acute Myocardial Infarction,” Brachytherapy 5(4):262-265; Davis, B. J., Bresnahan, J. F., Stafford, S. L., Karon, B. L, King, B. F., and Wilson, T. 2002. “Prostate Brachytherapy Seed Migration to the a Coronary Artery Found during Angiography,” Journal of Urology 168(3):1103; Davis, B. J., Pfeifer, E. A., Wilson, T. M., King, B. F. Eshleman, J. S. and Pisansky, T. 2000. “Prostate Brachytherapy Seed Migration to the Right Ventricle Found at Autopsy following Acute Cardiac Dysrhythmia,” Journal of Urology 164(5): 1661).

Means for counteracting migration of conventional seeds have been developed but call for linking (stranding, chaining) consecutive seeds with suture or the incorporation of a self-expanding bulb (see, for example, Bhagavatula, S., Thompson, D., Doninas, C., Haider, I, and Jonas, O. 2021. “Self-expanding Anchors for Stabilizing Percutaneously Implanted Microdevices in Biological Tissues,” Micromachines 12(4):404; Warrell, G. R., Xing, Y., Podder, T. K., Traughber, B. J., and Ellis, R. J. 2018. “Reduction of Seed Motion Using a Bio-absorbable [vicryl] Polymer Coating during Permanent Prostate Brachytherapy Using a Mick Applicator Technique,” Journal of Applied Clinical Medical Physics 19(3):44-51; Badwan, H. O., Shanahan, A. E., Adams, M. A., Shanahan, T. G., Mueller, P. W., Markwell, S. J., and Tarter, T. H. 2010. “AnchorSeed for the Reduction of Source Movement in Prostate Brachytherapy with the Mick Applicator Implant Technique” Brachytherapy 9(1):23-26).

That contemporary radioactive seeds can be implanted in structures other than the prostate as a guide to allow more accurate localization preceding resection to allow the removal of a smaller amount of tissue with the seed removed with the malignancy, such as preceding and thereafter succeeding a breast lumpectomy or the removal of a smaller volume of lung has been implemented since at least 2010 (see, for example, Hassing, C. M., Tvedskov, R. F., Kroman, N., Klausen, T. L. Djurhuus, S, and Langhans, L. 2016, Op cit.). Awareness of the utility of radioactive seeds to serve in different locations is becoming more apparent, and such uses have been expanded (see, for example, Bhagavatula, S., Thompson, D., Doninas, C., Haider, I, and Jonas, O. 2021, Op cit.).

Recognized at the outset in 1901, long before the usefulness of radioactive seeds as such to treat the prostate, sealed sources of radiation in other forms were used as intraparenchymal, or interstitial brachytherapy to treat malignancies in tissue other than, and often remote from, the prostate, such as in the breast and the cervix, or as having been situated beside the malignancy, plesiotherapeutic brachytherapy. Since then, the treatment of pancreatic and lung cancer have been added to the list (see, for example, Mayer, C. and Kumar, A. 2022. “Brachytherapy,” online, Treasure Island, Fla.: StatPearls Publishing; Rashid, A., Pinkawa, M., Haddad, H., Hermani, H., Temming, S., and 3 others 2021. “Interstitial Single Fraction Brachytherapy for Malignant Pulmonary Tumors,” Strahlentherapie and Onkologie 197(5):416-422; Cheng-Gang, L., Zhou, Z.-P., Jia, Y.-Z., Tan, X-L., and Song, Y.-Y. 2020. “Radioactive ¹²⁵I[odine] Seed Implantation for Locally Advanced Pancreatic Cancer: A Retrospective Analysis of 50 Cases,” World Journal of Clinical Cases 8(17):3743-3750).

Even without the addition of new means of implantation, the applications of conventional brachytherapy such as implemented with nominally prostate seeds has expanded to include other organs (see, for example, Ashida, R., Fukutake, N., Takada, R., Ioka, T., Ohkawa, K., and 5 others 2020. “Endoscopic Ultrasound-guided Fiducial Marker Placement for Neoadjuvant Chemoradiation Therapy for Resectable Pancreatic Cancer,” World Journal of Gastrointestinal Oncology 12(7):768-781; Ono, S., Ueda, Y., Ohira, S., Isono, M. Sumida, I., and 6 others 2020. “Detectability of Fiducials' Positions for Real-time Target Tracking System Equipping with a Standard Linac for Multiple Fiducial Markers,” Journal of Applied Clinical Medical Physics 21(11):153-162; Scher, N., Bollet, M., Bouilhol, G., Tannour, R., Khemiri, I., and 10 others 2019. “Safety and Efficacy of Fiducial Marker Implantation for Robotic Stereotactic Body Radiation Therapy with Fiducial Tracking,” Radiation Oncology 14:167; Vinogradskiy, Y., Goodman, K. A., Schefter, T., Miften, M., and Jones, B. L. 2019. “The Clinical and Dosimetric Impact of Real-time Target Tracking in Pancreatic SBRT [stereotactic body radiation therapy],” International Journal of Radiation Oncology—Biology—Physics 103( ):1268-275; Hassing, C. M., Tvedskov, R. F., Kroman, N., Klausen, T. L. Djurhuus, S, and Langhans, L. 2016. “Radioactive Seed Localization of Renal Cell Carcinoma in a Patient with Von Hippel-Lindau Disease,” Clinical Case Reports 5(1):26-28; Trumm, C. G., Haeussler, S. K., Muacevic, A., Stahl, R. Stintzing, S., and 5 others 2014. “CT Fluoroscopy-guided Percutaneous Fiducial Marker Placement for CyberKnife Stereotactic Radiosurgery: Technical Results and Complications,” Journal of Vascular and Interventional Radiology 25(5):760-768) and to exert traction on tissue to effect the dilatation of a stricture.

Accordingly, newer applications that anticipate the need for an expeditious means for placing tiny medicinal and irradiating implants in inaccessible locations such as from the lumen abaxially through the intima into the wall surrounding the lumen of a vessel quickly by means of a transluminal device, and/or in larger numbers than might be accomplished quickly by injection through a 19 or 2 gauge fine aspiration needle are coming, so that the means to support these applications should be made available beforehand.

### 3. Types of Spherule Insertion Tools

Spherule, or miniball, insertion tools are intended to considerably facilitate the placement of spherules whether in an open surgical field or endoscopically into the parenchyma of the target structure or neoplasm or into the wall surrounding the lumen of tubular bodily structure such as a blood vessel, the esophagus, the trachea, or the gut, for example. Detailed descriptive matter with accompanying drawing figures to detail the internal mechanisms of spherule insertion tools appears in the parent application hereto, namely Ser. No. 15/932,172, entitled Integrated System for the Infixion and Retrieval of Implants, readily available online.

Whereas divisional applications must address the different tools described in the parent application separately, the parent application discusses various interrelations among the tools described when used together as components as a related set of tools as components in a system, making a review of the parent application essential to convey an understanding of the system overall. For brevity and to avoid redundancy, the descriptive information pertaining to the constitution and uses of spherules and the tools provided to expedite the insertion of these in the parent application is incorporated here in its entirety by reference, citation and/or reiteration thereof herein used only when necessary.

Spherule insertion tools are of two types, those of the kind shown in FIG. 1 for use in an open surgical field and transluminally when the internal diameter or caliber of the ductus makes use thus safe, and those of the kind shown in FIG. 2 for use transluminally. Parent application Ser. No. 15/932,172, addresses the attributes and capabilities that distinguish each type in detail. With conventional means third, the three provide a set of tools of which each best serves a different function, so that used together allows each implant to be securely placed. In addition to the drawing figures provided here, additional drawing figures and detailed textual descriptions describing the internal mechanisms of spherule insertion tools appear in the parent application of which the content is incorporated here by reference in its entirety.

### OBJECTS OF THE INVENTION

To provide hand tools in suitable sizes capable of inserting small implants directly into the wall surrounding the lumen of any tubular anatomical structure in concentric relation to the wall thereof, as well as into any other tissue.

To dispel the difficulty in properly positioning small spherule implants manually one at a time so that the use thereof would be discouraged despite the considerable and versatile medical utility such implants have to offer, especially when used in combination.

To provide surgical hand tools that will facilitate the insertion of a train of small medicinal, radiation-emitting, magnetically susceptible, or magnetized spheroidal implants beneath the adventitia or outer tunic of a tubular anatomical structure or through the surface of an organ, gland, or lymph node, for example, to a shallow or greater depth thereby to make possible the application of a retracting force or stenting in cooperation with a surrounding, or perivascular, magnetized collar or subcutaneously positioned magnets.

To make possible the release of medication or radiation within the wall surrounding the lumen of a ductus whether a vessel, the trachea, esophagus, ureter, oviduct, or the duct of a gland, with or without the application of magnetic retractive force to dilate the lumen thereof, or beneath the outer tunic or deeply within the parenchyma of any organ, gland, or volume of tissue.

To allow the implantation of a neoplasm, regardless of its depth, within an organ, gland, or bodily ductus, with medicinal-releasing, such as chemotherapeutic, and/or a brachytherapeutic radiation-emitting implants.

To allow the release into the wall surrounding a blood vessel of a tissue strengthening agent to dispel the risk of a silent aneurysmal rupture in a patient with a congenital connective tissue disorder, for example, especially in an infant, thus allowing the deferral of a traumatic surgical correction, or if surgery is to be accomplished without delay, then with tissue more amenable to incision or clamping as necessary.

To facilitate the insertion of a train of miniature spheroidal, or miniball, implants wherein the internal organization in layers or subsidiary spherules of each spherule and the relative proportion of medicinal, irradiating, or magnetically susceptible content in each, as well as the depth to which each is inserted, is freely adjustable.

To facilitate the proper placement of miniballs of any type in any sequence and to any depth, and in so doing, significantly reduce the procedural duration, and when necessary, the amount of time the patient must be subjected to general anesthesia.

To allow remote access to deep tissue for the insertion of small miniball configured implants through a small, or ‘keyhole’ incision at the body surface, eliminating the need for more extensive incision, much less the need to create an open surgical field.

### SUMMARY OF THE INVENTION

This nonprovisional divisional application of parent application Ser. No. 15/932,172, entitled Integrated System for the Infixion and Retrieval of Implants, describes and illustrates the structure and function of surgical hand tools devised to allow the quick and properly positioned insertion of small spheroidal, or miniball implants uniquely conformed to be dispense by such a tool within tissue. The projectiles and tools for their insertion exclusively related to one another, the two together comprise a single invention. The insertion of a miniball into the wall surrounding a tubular anatomical structure, or ductus, or deeply into the parenchyma of an organ, gland, lymph node, or volume of tissue can serve any one of several or a combination of purposes. If incorporating magnetically susceptible matter such as soft iron-silicon, the implant can be drawn by a perivascular collar or subcutaneously positioned magnets as a source of tractive force to dilate a substrate ductus as an extraluminal stent.

When sought is the ability to retract an errant implant otherwise primarily containing medicinal or radioactive contents, for example, the amount of magnetically susceptible matter is less. If containing one or more medicinal substances, and/or sealed to emit local radiation over a relatively brief period as well, a miniball incorporating both will provide highly localized release in an absolute total amount tiny in relation to the body as a whole, eliminating the side effects associated with systemic dispersal.

### DESCRIPTION OF THE DRAWING FIGURES

FIG. 1 is a longitudinal section view of a simple pipe-type single barrel-assembly for use in a larger lumen such as the trachea or the esophagus, wherein the anatomy is structurally differentiated or where each miniball implant should be precisely located in relation to the lesion, shown with a deflection- or bounce-plate attachment for reversing the direction of the trajectory of a miniball at an angle equal and opposite to that of the initial impact against the bounce-plate, shown without side-clips for attaching a miniature cabled device such as an endoscope laser or laser.

FIG. 2 is a mid-longitudinal section through a 2- or 4-barrel-tube ablation and angioplasty-capable center-discharge muzzle-head with radial projection units and recovery electromagnets oriented or chambered normal to the long axis of the barrel-assembly, and equipped with an embolic trap-filter shown in FIG. 3 which is deployed or unstowed and retracted or stowed by means of the plunger solenoid at the bottom of the filter silo in the extended nose and radial projection units shown diagrammatically in FIG. 4 which are raised, that is, projected outward, by sending current through a thermal expansion wire at the base of each unit.

FIG. 3 is a diagrammatic detail view of an embolic trap-filter in the nose of a barrel-assembly muzzle-head showing the embolic trap-filter and the plunger solenoid used to deploy or unstow, or retrieve or stow, the trap-filter.

FIG. 4 is diagrammatic cross section view through a representative radial projection unit mounted about the outer surface of an angioplasty-capable barrel-assembly, each projected from the side, or deployed, by sending current through a thermal expansion wire at the base of each unit.

FIG. 5 provides a diagrammatical depiction of four differently configured working tips used in different abrading tool-inserts of which any such working tip or injection device is inserted into the radial projection unit to allow it to be lifted into working contact with the internal surface of the lumen or any other tissue.

FIG. 6 shows a simple control panel for a combination-form ablation or ablation and angioplasty-capable barrel-assembly such as shown in FIGS. 71 and 78 of the parent application Ser. No. 15/932,172, with heatable turret-motor stator, heat-windows, evidement radial projection units, independently heatable recovery or electromagnet windings, which allows the insertion of an excimer laser, directional or rotary burr atherectomizer, or a fiberoptic endoscope, for example, in the central canal.

FIG. 7 provides a diagrammatic representation of the control components and connections within the power and control housing of a combination-form ablation or ablation and angioplasty-capable barrel-assembly such as that shown in FIGS. 71 and 78 of the parent application, wherein each function is assigned to a separate rather than joint microcontroller, the onboard control panel shown in FIG. 6 which is same as that shown FIG. 79 of the parent application.

FIG. 8 shows a longitudinal section view of a gravity fed single barrel (single barrel-tube; monobarrel) interventional airgun which incorporates plural control points for adjusting the exit velocity over a range that allows its use for different tissues at different angles and to different depths in quick succession, shown with a plunger or dead-man type switch trigger.

FIG. 9 shows a diagrammatic representation of a semiautomatic positional control system for an interventional airgun, semiautomatic in this context meaning that the operator directs action by means such as the joystick shown in FIG. 9 which the system effectuates.

FIG. 10 shows a diagrammatic representation of the timing and positional componentry used to coordinate the automatic discharge as an auxiliary function and instantaneous positioning in transluminal displacement and rotational angle of the muzzle-head of an airgun such as those shown here in FIG. 8 and in FIGS. 81 and 82 of the parent application, but equipped with a rotary clip magazine such as those shown here in FIG. 1 and in FIGS. 31 and 32 of the parent application, for use with multiple barrel-tubes to allow the accurate implantation of miniballs in a close formation.

### DESCRIPTION OF THE PREFERRED EMBODIMENTS

The finer details of spherule implant insertion tool or discharge device structure are set forth in parent application Ser. No. 15/932,172, entitled Integrated System for the Infixion and Retrieval of Implants, which is readily available online. This divisional application will review the features of spherule implant insertion tool components to include pipe and torpedo-configured spherule implant discharge devices, or barrel-assemblies with the omission of the fine details thereof. Figure numbers higher than 10 appear in the parent application.

Open Monobarrel, or Simple Pipe Type Barrel-Assembly, with Bounce-Plate

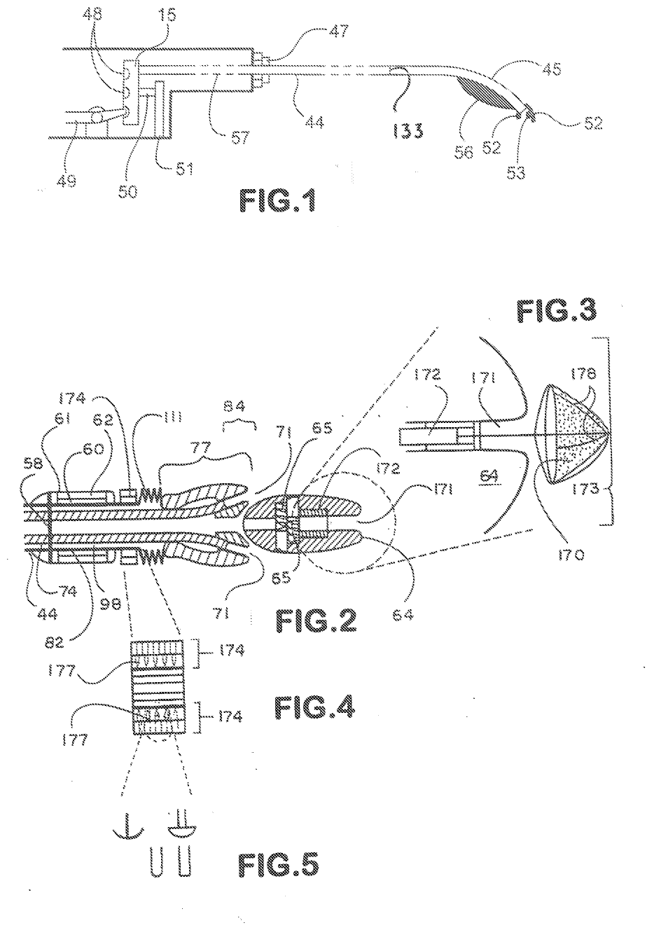

A barrel-assembly for use in the vascular tree must present a smooth and slippery surface, allow quick operation as one means for minimizing hypoxia, and incorporate features that minimize if not eliminate the need for withdrawal and reentry. This fact and the need to avoid any projections prompts enclosing the barrel-tubes of a radial discharge barrel-assembly in a slippery torpedo-shaped shell such as shown in FIG. 2 which is internally as well as externally protective in virtually eliminating gouges, incisions, abrasions, and perforations, improves tool positional stability, and strengthens the tool at its working end. A benefit in the use of a transluminal spherule insertion tool is that it eliminates the need for access to the insertion site through an incision at the body surface as do stays. Another advantage is elimination of the possibility of causing injury to the recurrent laryngeal nerve, of which the consequences can be dire.

Compared to the structurally differentiated trachea with its cartilage rings, the relatively undifferentiated gross structure of vascular, gastrointestinal, urogenital (urinogenital, genitourinary) and gamete transmitting ductus which pulsate or forcibly undulate during peristalsis can be secured by a larger number of miniballs at each level placed at different radial angles with less regard for precise placement. While a simple pipe or open barrel-assembly such as that shown in FIG. 1 is meant to implant no more than a single miniball at a time through the internal surface of a structured ductus, an enclosed, or torpedo-shell barrel-assembly can discharge up to four miniballs in different radial angles at the same level along the ductus with each discharge.

By contrast, the placement of miniballs in the trachea must be discretionary such that each miniball can be aimed at a specific point. This is more easily accomplished using a single barrel single discharge tool such as the simple pipe or open barrel-assembly shown in FIG. 1 which not enclosed within a torpedo shaped shell such as the discharge device or barrel-assembly shown in FIG. 2 can be directly viewed. A finely-gauged fiberoptic scope or angioscope and laser sight or pointer clipped alongside the insertion tool assist to provide a view of the target site and indicate the aiming point for implantation that allow each discharge to be accurate.

FIG. 31 in the parent application shows a simple pipe type barrel-assembly without a bounce-plate (deflection-plate, ricochet-plate; rebound-tip; rebound-plate, rebound deflection-plate, rebound angle deflection plate), and FIG. 1 here, which is the same as FIG. 32 in the parent application, shows the same basic device but with the addition of an attachable bounce-plate, the embodiment shown omitting an intracorporeally deployable and retractable bounce-plate mechanism fastened along the upper surface toward the distal end of the pipe as shown in FIG. 35 in the parent application.

Where to rotate the tool might injure tissue near the discharge end or muzzle of the tool, a bounce-plate allows the trajectory of a miniball to be reversed. Whereas the positioning of miniballs along the intima of an artery with its lack of distinguishable structure from one level to the next is noncritical, to place miniballs in a structured ductus must be undertaken with precision. For this reason, unless preceded with considerable practice, the use of a bounce-plate inside a structured milieu is to be discouraged.

In FIG. 1, the portion of the barrel-assembly distal to the barrel-catheter 44 is the muzzle-head 45. Optionally, to allow slight shifts or deviations in the aiming point or the point at which the miniball will penetrate the target tissue, the proximal face of a bounce-plate which is struck can be formed or ground with a concave contour. The concavity can be along the vertical or the horizontal axis or both. The controls provide calibrated gauges to support this capability. Unlike a radial discharge barrel-assembly, which can only be rotated over an are determined by the eventual twisting of its barrel-tube or tubes, rotatory joint 133 allows muzzle-head 45 to be rotated endlessly.

In a barrel-assembly for use without a bounce-plate or with a bounce-plate attachment described below, rotatory or swivel joint 133 must be placed at a level sufficiently proximal to allow muzzle-head 45 to be rotated in relation to the barrel-catheter. In a barrel-assembly with an intracorporeally controllable bounce-plate mechanism described below, rotatory joint 133 must also be placed sufficiently proximal to allow clear access to the bounce-plate mechanism controls, which must be mounted to the top of muzzle-head 45 with which the mechanism rotates.

Provided muzzle-head 45, which is inflexible from the distal (front) end to rotary joint 133 and includes a moderate curvature toward the front end with recovery electromagnet 46 shown in FIG. 33 of the parent application within magnet housing 56 nested therein can be passed through the vocal folds and larynx, for example with little risk of injury, rotary joint 133 will join muzzle-head 45 to barrel-catheter 44. However, if the inflexibility of the muzzle-head interferes with safe clearance, then a tool of the same kind having a working end with a less pronounced curvature is used.

Provided the ductus can be negotiated from within the lumen without injury and the implants properly positioned, to approach from outside the ductus through one or more small incisions at the body surface in order to use stays is to be discouraged. For use in an open surgical field, a simple pipe type or open monobarrel barrel-assembly such as shown in FIG. 1 can incorporate a curve toward the discharge end which varies between pronounced to omitted so that the barrel is completely straight.

Omitted here but shown in detail in FIGS. 35 thru 37 of the parent application, the materials of a bounce-plate mechanism must be flexible to comply with that of the barrel-catheter, which is readily accomplished with many different materials using a bevel gear but not a pulley arrangement within control tube 135 shown in FIG. 37. The division of the control sleeve into a proximal control slide-box portion 134 and distal controlled slide-box portion 140 is due to the requirement for control rod 135 to be rotatable, hence, cylindrical, whereas the distal bounce-plate 53 must have bilateral extension. Continuous sleeve or separate slide boxes 134 and 140 can be made of any suitable nonmagnetic metal or plastic with all corners and edges rounded or blunted.

Control slide-box 134 in FIGS. 35 and 36 of the parent application Ser. No. 15/932,17, is lined with a material such as felt that imparts a smooth sliding action to slide-block 137, while controlled slide-box 140 is lined with an absorbent material such as gauze or a nonallergenic foam which can be wetted with a mucolytic such as acetylcysteine or a mucinolytic (mucinase, mucopolysaccharidase). The absolute amount of acetylcysteine is too small to cause stomatitis or induce nausea or rhinorrhea; however, to prevent bronchospasm, only light wetting is used in the distal airway. When made continuous, the proximal lumen must be circular to allow control tube 135 to be rotated, while the lumen of the distal controlled slide-box 140 must be horizontal to accommodate bounce-plate 53.

Thus, a lumen uniform in diameter or gauge from end to end would have to equal the width of bounce-plate 53, which will most often equal the diameter of exit-hole 55. This would double the cross-sectional area of muzzle-head 45 and bounce-plate mechanism combination. Fundamental objects being to minimize the muzzle-head to miniball diameter ratio and cost, separate proximal and distal lumina or slideways are provided as control slide-box 134 and controlled slide-box 140. The proximal end of bounce-plate control slide-box 134 then spans across a nonrotatable and intracorporeal joint between the inflexible muzzle-head 45 and the distal end of the barrel-catheter 44. Rotary joint 133 must then be sufficiently proximal to allow the barrel-assembly distal to it to be rotated and the bounce-plate to be controlled.

Since the bounce-plate mechanism does not span over rotary joint 133, the muzzle-head remains endlessly rotatable. Bounce-plates range in cost of manufacture and precision from a simple attachment to a built in precision mechanism that allows the discharge to be redirected without the need to move, much less remove, the muzzle-head from the body. Unless the insertion tool is completely straight, located in the concavity on the underside of the muzzle-head in FIG. 1 here is the recovery electromagnet 46 shown inside magnet housing 56 in FIG. 33 of the parent application. Rather than ejected perpendicularly to the surface of the lumen wall, the miniballs are delivered at an acute angle to be seated subadventitially or subfibrosally, or if necessary, medially or submedially (superintimally).

Enclosed Multiple Barrel Barrel-Assembly

Insertion thus seeks to wedge the miniball in place and avoid pull-through or delamination of tunic layers which may have been weakened by disease. In the data gathering through actual testing such as described in section XVII of the parent application entitled Testing and Tests that precedes any such procedure with an enclosed multibarrel muzzle-head as shown in FIG. 2, the internal and external elastic laminae within the walls of larger arteries facilitate finding the correct discharge velocities to position miniballs. Discharge at an acute angle avoids a singular vector trajectory that normal to the intimal surface, would be more prone to rebound, possibly back into the lumen, if not perforate through the adventitia.

In FIG. 2, part number 74 is a barrel-tube, meaning one of the tubes through which the spherules or miniballs are propelled up to ejection through muzzle-ports, or exit holes 71, part number 61 is the through-bore turret-motor housing, and 62 the turret-motor stator. Part number 60 is the direct drive, or cogless, brushless, hollow shaft, or through-bore rotor of the turret-motor. FIG. 2 shows one of several embodiments similar in construction. The channel separating barrel-tubes 74, or the central canal, allows different miniature fiber cable devices such as a scope, laser, or intravascular ultrasound probe to be interchangeably advanced and withdrawn through the center of the device up to the working end midprocedurally.

Relegating a more detailed description of the structure to the parent application, only the distal segment of barrel-catheter 44 separated to create a rotary joint and journaled within through-bore rotor 60 of the turret-motor rotates with rotor 60, and since muzzle-spindle 77 is attached to the distal end of the distal end of the distal segment, spindle 77 is rotated. Part number 111 is a flex joint to divert the muzzle-head when continued advancement directly forward would injure the endothelial or urothelial lining if not the intima of the surrounding luminal wall, and 174 is a token radial projection unit with any of the tools having interchangeable working tips such as those suggested in FIG. 5.

Not shown here are radial projection catheters, angioplasty tools without spherule ejection capability that incorporate many radial projection units with various tool inserts to treat the luminal wall. For a more detailed description of both barrel-assemblies such as that shown here in FIG. 2 and radial projection catheters, refer to the parent application.

For structured lumina or where for any reason it is advisable to reverse the direction of miniball entry, such as when entry orients the muzzle-head retrograde so that the passage of contents might eventually urge the miniball back into the lumen, a means for reversing the direction of entry is desirable. While in some instances, such as for treating the trachea in a very small or ‘teacup’ sized dog where a simple pipe is too large for safe passage, an endoluminally deployable and retractable bounce-plate mechanism of a types now to be described can be added to a small gauge monobarrel radial discharge barrel-assembly.

However, radial discharge barrel-assemblies are ordinarily intended for implanting substantially uniform gross anatomy as in virtually every other type of ductus (ureters, gastrointestinal tract, arteries) where exactitude in aiming is noncritical and multiple miniballs can be discharged simultaneously. To allow a clearer view of the exit-hole (muzzle), aiding aiming accuracy in relation to the differentiated structure within the trachea, simple pipes omit a shell (body, enclosure).

Barrel-assemblies for use within vessels, for example, provide a shell about the muzzle-head to protect the lumen wall, and are intended primarily for veterinary use to alleviate tracheal collapse, although the same conformation makes simple pipes more suited to applications outside of lumina than radial discharge barrel-assemblies. Since a simple pipe barrel-assembly with the endoluminally deployable bounce-plate mechanism shown in FIG. 36 of the parent application is passed through the larynx and down the trachea if not a bronchus with the bounce-plate retracted, a barrel-assembly that is equipped with an endoluminally controllable bounce-plate mechanism also reduces the risk of injury and thus the duration of general anesthesia.

Simple pipes can be made 1. To accept a simple bounce-plate attachment that to slip on and off requires removal from the patient and to change the vertical angle (elevation) requires bending and the rotational angle requires rotation of the muzzle-head; 2. With a substantially fixed rebound angle bounce-plate that can be slid forward into position or deployed and rotated while endoluminal whenever needed; or 3. So that vertical and rotational angular adjustments are under positive or direct mechanical control and calibrated for precision. The latter two are adjustable in downward inclination (elevation) of the bounce plate and thus the angle of rebound along the vertical axis of the muzzle-head.

By providing a calibrated control arm, that rotatable thus allows fine adjustment in the angle of discharge through a wide radially and longitudinally arcuate volume to its underside. When the muzzle-head can be long enough to extend outside the body, the forcibly bendable metal muzzle-head of the barrel-assembly is joined at its proximal end to the barrel-catheter by an internally smooth rotary joint for rotation as a handpiece. The intracorporeally adjustable bounce-plate configurations shown in FIGS. 35 thru 37 in the parent application are then mounted to the upper surface of the muzzle-head. If not, the control spans over the junction between the muzzle-head and barrel-catheter. Either configuration allows the bounce-plate to be controlled while the barrel-assembly is in use to more quickly achieve fine control over the angle of discharge.

To reduce the risk of injury, permanent endoluminal bounce-plate control mechanisms must be as little protrusive as possible and have outer edges and corners that are blunted (rounded, curved). The use of a bounce-plate pertains only to simple pipe, not radial discharge type barrel-assemblies. The forward edge or rim of the pipe is generally cut an angle to allow flush placement against tissue to be implanted. The overhang generally accommodates the reverse discharge of miniballs when a bounce- or rebound deflection plate is attached. To minimize accidental injury, the tip of a simple pipe is covered with an elastomer guard. Protruding beyond the tip of the simple pipe, an overhang or roof-configured bounce-plate especially requires a protective guard.

Likewise for flush abutment, the tip of a walled-around bounce-plate is angled with the tip directed in the opposite direction. Walled-around, Krummlauf-type continuously curved barrels, and hybrid versions of the two for reversing the direction of discharge would allow good control over the trajectory but are impractical because of the enlargement if not hook conformation at the distal end of the pipe. Such ends make insertion and withdrawal difficult and more prone to cause laryngeal injury. The bounce-plate can be a friction-fitting attachment or a permanent feature. Because to observe the muzzle-port or ports is unintended and more difficult with a barrel radial discharge barrel-assembly, the simple pipe is preferred for use in the anatomically differentiated airway.

A torpedo-enclosed single barrel radial discharge barrel-assembly is used only in the airway of the smallest dogs with collapsed trachea where there is not the space to manipulate an open simple pipe such as shown in FIG. 1 and in distal segments of the bronchi where these are undifferentiated. For accurate implant placement in structured anatomy, the insertion tool has a viewing scope attached at its side. The structural differentiation and consequent need to place the implants in a discretionary manner can in some cases recommend the availability of a simple pipe barrel-assembly with bounce-plate (deflection-plate, ricochet-plate; rebound-tip; rebound-plate), which allows reversing the direction of the trajectory, that is, directing the miniballs back toward the operator or proximad.

This capability can be beneficial, for example, in the trachea to introduce implants into the posterior junction of each successive cartilage ring with the annular ligament, as described below. Published testing results on specimens having just died are consulted before engaging upon such use. The avoidance of withdrawal and reentry in the airway is not onerous as it is in the bloodstream, and such a capability is often unnecessary. The single barrel radial discharge barrel-assembly and not the simple pipe is recommended when space is lacking to insert and withdraw the simple pipe without risk of injury to the larynx.

In smaller patients, a simple pipe barrel-assembly may be usable for a distance towards the bronchi, down to which the diameter of the lumen becomes so restrictive that it becomes necessary to withdraw and replace the simple pipe with a single barrel radial discharge barrel-assembly. The bounce-plate is thus incorporated into a second simple pipe rather than as an option that would be opened or closed in a single embodiment. Under such circumstances, withdrawal and reentry is preferable or essential, so that a single embodiment capable of discharge both distad and proximad, which to provide entails additional complexity and cost greater than the sum for separate barrel-assemblies where one does and the other does not have a bounce-plate, is not preferred.

Accordingly, a simple pipe barrel-assembly that reverses the direction of the trajectory is provided in a separate barrel-assembly. FIGS. 32 and 34 of the parent application show a simple pipe barrel-assembly with a manually attached bounce-plate at the distal end of the muzzle-head. An attachable bounce-plate is not deployable or retractable with the muzzle-head intracorporeal and is suitable only for occasional or isolated use for directional reversal of the trajectory when laryngeal clearance to admit the tip with bounce-plate is adequate.

Bounce-plates that allow insertion through the larynx then extension of the bounce-plate into position during use and retraction before withdrawal are described below in this section and shown in FIGS. 35 thru 37. Except for the addition of bounce-plate 53 and a soft protective annulus 52 adapted for the change in configuration of the muzzle-head that results from the bounce-plate, the simple pipe is the same as that shown in FIG. 31 with only a soft rubbery protective ring 52 surrounding the tip. Since the front portion of a full circle protective annulus 52 interferes with mounting bounce-plate 53, a hybrid annulus consisting of a soft or rubbery portion at the rear and bounce-plate portion at the front provided.

The bounce-plate portion may consist of bare metal or metal with an outer rubbery coat, which is then preferably unitary with the rubbery rear portion. A detailed view of the tractive electromagnet 46 mounted in the concavity on the underside of the simple pipe barrel-assembly in the curve 45 approaching its distal end is shown in FIGS. 31 thru 34. The loss of a miniball in the airway being unlikely and posing little risk even were it to occur, an antemagnet chamber as seen in magnet assemblies used in radial discharge barrel-assemblies for use in the bloodstream described below, is not used. In FIG. 33, recovery electromagnet 46 is enclosed within electromagnet housing 56 made of any hard plasticizer free resin and bonded in position by means of an adhesive that is pliable after curing as discussed in the preceding section.

When the airway is large enough that withdrawal of a muzzle-head without bounce-plate as shown in FIGS. 31 and 33 and reentry with a rebound muzzle-head or muzzle-head having a bounce-plate, such as those shown in FIGS. 32 and 34 poses minimal risk of injury, the separate embodiments are used. When the airway is not so small as to necessitate the use of a radial-discharge barrel-assembly, a simple pipe with a bounce-plate is used. The bounce-plate is a distal tip cap (crown, ferrule) friction-secured fitting that to attach or replace necessitates removal and reintroduction of the pipe; a bounce-plate that is endoluminally deployable and retractable addressed in the next section, and one that is endoluminally adjustable in angle addressed in the section following that.

If, for example, the simple pipe is polypropylene on the outside and the nonferrous metal of the bounce-plate is an alloy of aluminum, the adhesive, which must remain pliant after cured, is preferably a two part polyurethane, such as Loctite U-05FL, mentioned above in the preceding section entitled Simple Pipe Barrel-assembly. The angle of rebound equal and opposite to the angle at which the miniball strikes the bounce-plate upon exiting the original muzzle-port, seen in FIG. 34 as 55, the angle described between the trajectory upon colliding and rebound off of the bounce-plate is usually 45 degrees.

If nonrotatably mounted to the airgun muzzle at the twist-to-lock connector, then rotary joint 133 shown here in FIG. 1 and in FIGS. 31 thru 33 of the parent application is used. If connected to an airgun mounted to a linear positioning stage on a swivel carriage as shown here in FIG. 9 corresponding to FIG. 83 in the parent application, then a second point for rotation is available. The intracorporeally controllable bounce-plate mechanisms described in the following sections provide an additional adjustment for the rotational angle of rebound. Similarly, with an air pistol, the barrel-assembly is rotated as a whole or at joint 133. When rotated midprocedurally, the ‘top’ or ‘upper surface’ of a barrel-assembly is that of the muzzle-head, not that of the bounce-plate or the vertical axis.

Whether along a longer muzzle-head, the junction between barrel-catheter and muzzle-head, or at a level along the barrel-catheter, rotary joint 133 must be positioned sufficiently proximal along the barrel-assembly that it remains extracorporeal and accessible for manual adjustment. Since the bounce-plate mechanism must be continuous and its push-pull handle and rotation lever or arm 138 shown in FIGS. 35 thru 37 must also remain accessible, rotary joint 133 must be positioned proximal to the proximal end of the bounce-plate mechanism. Control slide-box or sheath 134 is ordinarily extended up to controlled slide-box or sleeve 140. Controlled slide-box 140 is advantageously identical to distinct bounce-plate 53 housing when bounce-plate 53 is of a shape that would result in added expense were it unitary or continuous with the bounce-plate mechanism proximal to it.

Rebound dissipates the kinetic energy and momentum or propulsive force imparted to the miniball necessitating adjustment of the airgun setting. Since the simple pipe barrel-assembly is intended for use in the trachea and the single-barrel radial discharge barrel-assembly for use in the tracheobronchial tree when the lumen diameter is confining, sections to follow the description of these single barrel barrel-assemblies will be directed to the application of these barrel-assemblies for use in the airway. Multiple discharge barrel-assemblies, which are not used in the airway but rather in vessels and ducts are described later.

The simplest type of rebound deflection plate, shown in FIG. 1 here and in FIGS. 32 and 34 of the parent application consists of an angled tip that is slipped over the distal end of a pipe such as that shown in FIGS. 31 and 33 of the parent application after pulling off the rubbery ring intended to protect surrounding tissue in the larynx and surrounding the lumen from gouging. Once introduced, it is not retractable and therefore suitable when insertion is unlikely to be repeated. The type shown in FIG. 36 is deployable and retractable, and that shown in FIG. 37 rotatable as well to adjust the radial angle of discharge more finely than is readily accomplished by rotating the muzzle-head as a whole while the muzzle-head is intracorporeal (inside the body).

Closed Multiple Barrel Barrel-Assembly Radial Projection Tools

Indicated as part number 174 in FIG. 2 here but shown and described in detail in FIGS. 52a, 52b, 54, and 59 of the parent application, as exemplary for the two differently controlled types, tool-insert receiving units include a lift-shaft 182 containing tool-insert holding and lift platform 176 and are situated about the periphery of muzzle-head as 174 in FIG. 2 with callout provided in FIG. 4 between the front of turret-motor housing 61 to the rear, and elastomeric segment of convoluted tubing that serves as flex-joint (flexible joint) 111 to the fore, for example.

Tool-inserts can deploy variously configured curettage (evidement, scraper abrader-type) working tips for dislodging and relegating to the embolic filter 173 atheromatous or adherent crystalline matter or injectors, for example, four working tips diagrammatically represented in FIG. 5. In FIG. 2, part number 174 is a radially projectable tool which can be deployed by sending current to a thermal expansion wire 177 at its base to allow the barrel-assembly to be moved back and forth to remove plaque by evidement. The parent application Ser. No. 15/932,172, describes radial projection catheters, which independent of a barrel-assembly, consist of numerous such small lift tools arranged in immediately consecutive relation to accomplish thermal or abrasive angioplasty, for example.

Enclosed Multiple Barrel Barrel-Assembly with Integral Embolic Filter

The closed multiple barrel barrel-assembly shown in FIG. 2 provides a central canal which can convey the working end of a miniature cabled device such as an angioscope or intravascular ultrasound probe to the insertion site. Depending upon the application, the embolic filter (filter-trap, trap-filter, run-ahead filter) 173 shown in FIG. 3 as parachute, dragnet, or umbrella-shaped might just as easily be windsock, drag, or trawler type fishing net-shaped. Part number 172 is a push, or plunger, solenoid used to deploy and retract run-ahead embolic filter 173.

In FIG. 2, note that a clear channel 171 courses through the muzzle-head. FIGS. 2 and 3 show this channel as having been used to stow and unstow the embolic filter shown in FIG. 3. However, this same channel, or central canal, 171 can be used to pass through any miniature cabled device, whether an angioscope, laser, intravascular ultrasound probe, or rotary atherectomizer. Moreover, these can be inserted, retracted, and replaced with another miniature cabled device at any time during the procedure.

Angioplasty and Stenting Control Panel

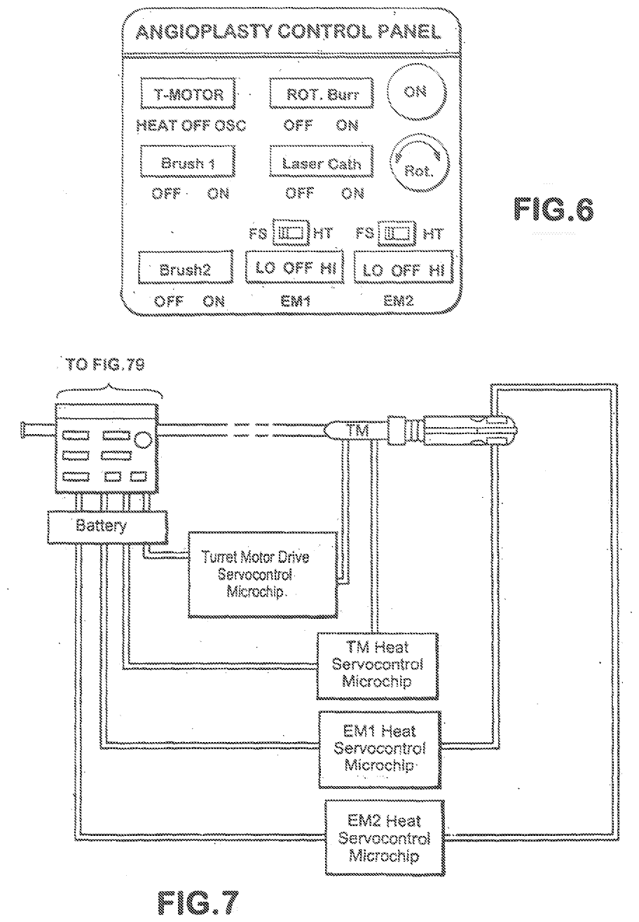

In barrel-assemblies for use in lumina that are less restrictive, the primary can include electrically and fluid controlled heat-windows and tool-inserts, and tool-inserts of either type may incorporate internal functions such as warming the contents of an injection syringe, which require additional circuits. Accordingly, the specific controls and the apportionment of these in ablation or angioplasty control panels of different barrel-assemblies and radial projection catheters vary considerably. For this reason, the control panel shown in FIG. 6 can be no more than exemplary. In a fluid circuit, current is used to control the electrohydraulic or electropneumatic control valve in each circuit fluid supply or pipe line. An angioplasty-capable barrel-assembly incorporates an angioplasty control panel such as shown in a generalized form in FIG. 6, the features shown described in the parent application.

Positional and Therapeutic Control Components in a Fully Capable Barrel-Assembly

FIG. 7 provides a diagrammatic representation of the control components and connections within the power and control housing of a combination-form ablation or ablation and angioplasty-capable barrel-assembly such as shown in FIGS. 71 and 78 of the parent application, wherein each function is assigned to a separate rather than to a joint microcontroller, the onboard control panel shown in FIG. 6 the same as that shown in FIG. 79 of the parent application.

In FIG. 7, TM stands for turret-motor, which can be used in two distinct modes, to have current passed through it in order to generate heat, and for the more conventional function of controllably rotating the muzzle-head, and EMs 1 and 2 stands for the electromagnetic windings in the muzzle-head, intended as standby recovery means for an errant miniball, but which likewise can have current passed through them in order to generate heat.

A fully featured barrel-assembly control system fully automates lumen preparation and therapy such as thermal, as well as exercise positional control of the barrel-assembly antegrade-retrograde, the rotational angle of the muzzle-head turret motor, and the deployment of accessories such as an embolic filter and radial projection tools. Automatic control in this regard is always semiautomatic in that control overall is never entrusted to the control system but rather administered by the operator or an assistant through the use of a joystick such as shown in FIG. 9, for example.

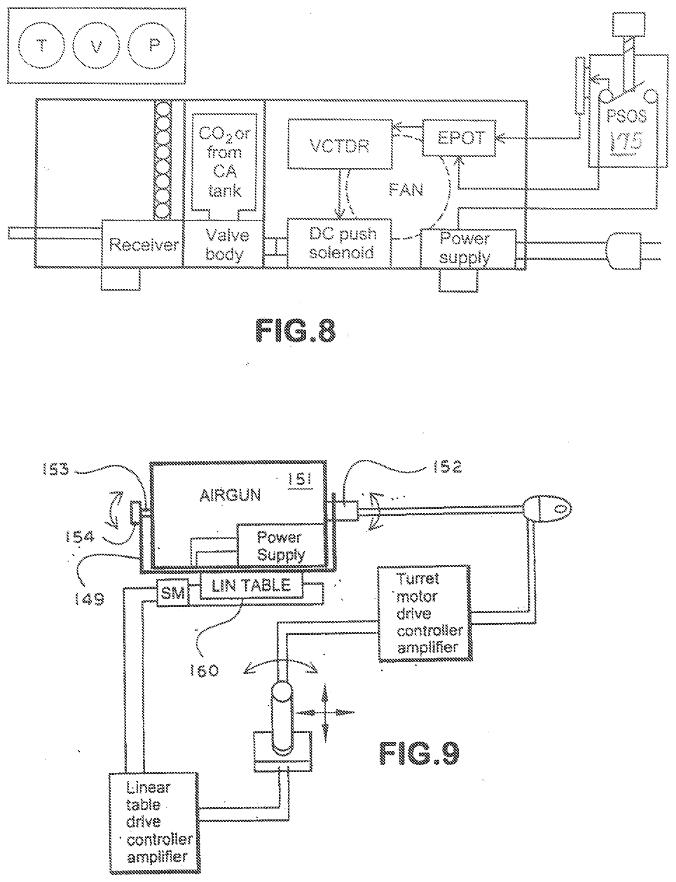

Interventional Airguns

Whereas barrel-assemblies conduct miniballs to the target and usually incorporate additional means for treating the internal surface of the lumen, the force to propel the miniballs is provided by a CO₂ or compressed air-powered, specially designed, that is, dedicated, interventional airgun such as those shown in FIG. 81, reproduced here as FIG. 8, and FIG. 82 of the parent application. FIG. 8 shows a longitudinal section view of a gravity fed single barrel (single barrel-tube; monobarrel) interventional airgun with plural control points for adjusting the exit velocity over a range that allows its use for different tissues at different angles and to different depths in quick succession, shown for actuation by the operator or an assistant by means of a plunger or dead-man switch type trigger.

In a simple embodiment intended for use with a refillable cylinder of compressed air that has been pressurized for use with a tissue of certain properties as shown here in FIG. 8 corresponding to FIG. 81 in the parent application, an electropneumatic valve consisting of a plunger solenoid actuator and valve body is used to admit and within a small range compared to a regulator, control the pressure of the gas used to propel each shot, hence the exit velocity and force of impact.

A dedicated interventional airgun can be a gravity fed monobarrel such as those shown here in FIG. 8 and FIGS. 81 and 82 of the parent application, or equipped with a rotary clip magazine such as the interventional airgun shown here in FIG. 1, and in FIGS. 31 and 32 of parent application Ser. No. 15/932,172, for use with multiple barrel-tube barrel-assemblies, which advances, withdraws, and rotates the muzzle-head in coordination with discharge to allow the uniform implantation of miniballs in close-formation. If connected to an airgun mounted to a linear positioning stage on a swivel carriage as shown in FIG. 9, then a second point for rotation is made available.

A control knob is provided to adjust the voltages that regulate the extent and duration that the electropneumatic valve opens to the pressurized gas. Providing dedicated interventional airguns with an additional foot control switch to trigger discharge is not preferred. A conventional electrical foot-switch must be adapted to incorporate a safety pin that must be released by depressing a lever with the toe of the opposite foot, and limited to triggering only, the foot switch is too limited. The incorporation into a foot operated control panel of all the controls necessary to use the apparatus is rejected as inviting unintended actuation.

Accordingly, FIG. 8 here corresponding to FIG. 81 in the parent application is a block diagram, not to proportion, of a gas-operated surgical miniball implant insertion airgun with compressed gas cylinder connected directly to the valve body inlet. While represented this and the dedicated interventional airgun next to be described are represented as gravity fed as suited to use with a simple pipe barrel-assembly, it is to be understood that either can also use rotary magazine clips and so accommodate any kind of barrel-assembly. In addition to the valve controls provided, different delivery tubes friction fit to the end of the barrel can be used to variously reduce the barrel exit velocity, hence, the force of impact.

Such an embodiment, using a single cylinder of compressed gas without the additional expense of a regulator, is suitable for use where the a range of exit velocities or forces of penetration is required, as when treating a single tissue to a single depth. Under normal circumstances, a disposable delivery catheter designed for the particular application is provided. A device as shown above and in the following FIG. 82 allows continuous variability in the force impact, which expedites testing tissues for the purpose of disposable catheter design. The compressed gas can be supplied, for example, from either an internal prefilled disposable CO₂ or by means of piping from an external CA compressed air cylinder.

Whereas CO₂ delivers 837 psi at 70 degrees Fahrenheit, a compressed air cylinder can be filled to a preferred pressure. With the interposition of a small adaptor, either a CO₂ or CA cylinder can be connected to the valve body inlet. Using a single source of compressed gas without regulator keeps the design simple and economical. A small CO₂ cylinder inserted within the enclosure makes the single-purpose airgun self-contained and compact. Containing nonliquified gas, a compressed air cylinder is larger and therefore connected from outside through a hose but can be filled to any pressure within its design specification. With or without a regulator, control with a single source of compressed gas is limited to reduction in the outlet pressure (also referred to as a canister or tank).

With this basic design, variability in shot impact force is limited to adjustment in the field strength and duration of plunger solenoid actuation. Preserving this simplicity and economy limits the pressure-reducing features that can be built into the airgun. Nevertheless, by connecting compressed air cylinders filled to different pressures, even the simple airgun can be used to treat different tissues to different depths of penetration. In such use, multiple cylinders of compressed air are connected and switched among manually by means of a pneumatic or electronically by means of an electropneumatic station valve.

This can be done at no great expense when switching is manual; however, the parts necessary to switch among different cylinders with electronic valves loses the economic edge over a design that affords continuous variability in pressure through the use of a regulator. A warming jacket containing a heating element or coil about the gas delivery tube with thermostat or pyrometer control can be used to change the temperature and so adjust the pressure.

Since conventional CO₂ cylinders are rated for up to 1800 pounds per square inch (psi), the range of pressure control gained in this manner is much less than it is with compressed gas cylinders, which can withstand pressure of thousands of pounds per square inch. For clarity, FIG. 8 here corresponding to FIG. 81 in the parent application and FIG. 82 therein show the pressure gauge P, temperature gauge or pyrometer T, and voltmeter V housed separately from the table-top or stanchion-mounted main unit. PSOS is a full-wave rectified regulated power supply output switch.

Continuing with FIG. 8, the take-offs for the different components are voltage divided by a bleeder resistor, each circuit controlled by a variable resistor. EPOT is an electronic potentiometer remotely operated from the remote hand control. In a simpler version, the potentiometer is mechanical, in the same position in the circuit, but mounted on the chassis rather than the hand control, and VCTDR is a voltage-controlled time-delay relay. Essentially, there are two circuits, one pneumatic, the other valving the passage of gas through the pneumatic circuit. The combination of the plunger solenoid and the gas valve constitute a special purpose impulse-actuated electropneumatic valve.

Whereas an enclosure-mounted manually operated potentiometer is less costly and assumes operation by an assistant, an electronic potentiometer in the remote hand control affords the operator direct control; both can be connected in series. Depressing the remote control ‘dead-man’ or plunger type trigger switch at the top of the joystick control connects the power supply through the EPOT and VCTDR to the undamped direct current powered plunger solenoid, energizing the solenoid coil. This causes the solenoid plunger (slug, armature) to strike or punch the spring-loaded valve inlet pin forcing open the valve within the valve body for the interval set by the VCTDR.

Use of the plunger switch trigger requires release of the safety by retracting a pin intromitted into the side of the control button or key which is placed at one end of a spring-loaded lever retracted by pressing the opposite end with the ball of the index finger. The gas thus admitted to the rear of the mini ball implant in the receiver propels the implant as a projectile through the barrel and delivery tube at the target tissue.

Adjustment in the output of the power supply through the potentiometer varies the actuation field strength of the solenoid, varying the punching force of the solenoid plunger against the valve pin.

Increasing the force of plunger impact upon the valve pin also slightly increases pin excursion, hence, valve open-time. Valve open-time is thus determined both by the interval that the switch connects the solenoid to the power supply and by the voltage. This timing may be controlled as a structural or mechanical feature of the switch contacts or through a separate electronic time-delay relay. Absent such a solenoid actuation time mechanism, the solenoid plunger would not retract until the switch was released, which interval is too long.

The discontinuous character of the function, which involves the intermittent discharge of sudden shots, does not lend itself to servomechanical control; instead, a V voltmeter indicating EPOT output on the enclosure serves to implement human feedback. The acrylonitrile butadiene styrene (ABS) enclosure with a thermal conductivity between 0.14 and 0.21 watts per meter-Kelvin (W/mK) and 97 cubic feet per minute (cfm) fan with plastic vanes and frame prevent the undesired buildup of heat that could materially alter the gas pressure and therefore terminal ballistics. Adjusting the fan speed and thus the volume of air moved through the enclosure by means of a thermostat is another way that the temperature of the gas can be controlled to obtain variability in pressure.

Conventional means exist for preventing the temperature to exceed a set limit, and even were such to malfunction, all compressed gas cylinders incorporate a pressure relief mechanism. Thus, even using a single cylinder, and even when the cylinder contains CO₂, which is not normally viewed as affording variability in pressure, numerous variables are available to control the pressure and therefore the force of impact and depth to which the shot will penetrate given tissue. Of these, the least costly embodiment shown here employs those variables that govern valve open time. In an embodiment that must afford a wide range of penetration forces for a single procedure, a regulator capable of continuously adjusting the gas pressure is used.

Whereas a regulator and the control means shown allow pressures less than that to which the cylinder is pressurized, increasing the temperature allows the cylinder pressure to be exceeded. The power controlled from the remote control hand piece is represented as controlling both the output from the power supply through the electronic potentiometer and the input power proportional time delay. That is, the same potentiometer is used to vary the input to the solenoid and the time-delay relay to continuously vary the force and interval that the valve is held open, both of which factors increase valve open-time. Separate control of the time delay does not significantly extend control variability.

Whether manually adjusted in a simpler model or electronically in one more costly, a regulator is usually controlled separately. In such an embodiment, the regulator is in effect the gross adjustment, whereas the controls shown here serve for fine adjustment. To avert disruption due to malfunction, more than one such relatively simple apparatus, each adjusted to the same settings, should be present. If more than two are available, differently adjusting these in pairs allows treating different tissues. An interventional airgun for suitable for procedures involving the treatment of different tissues to different depths in quick succession with redundant points of control to adjust the exit velocity is described in section XIII3c and shown in FIG. 82 of the parent application.

Control of Translational Motion of the Airgun and Rotation of its Turret-Motor

FIG. 9 us a pictorial schematic of the mounting of an interventional airgun such as that shown in FIG. 8 to allow advancement and retraction of the muzzle-head and rotation of the turret-motor along the lumen of an artery, for example. As shown in FIG. 2, this controls all functions of the barrel-assembly, to include the deployment of radial projection tools of which only one is shown in FIG. 2 to sweep away noncalcified atheromatous lesions which are then caught in embolic filter 173 in FIG. 3.

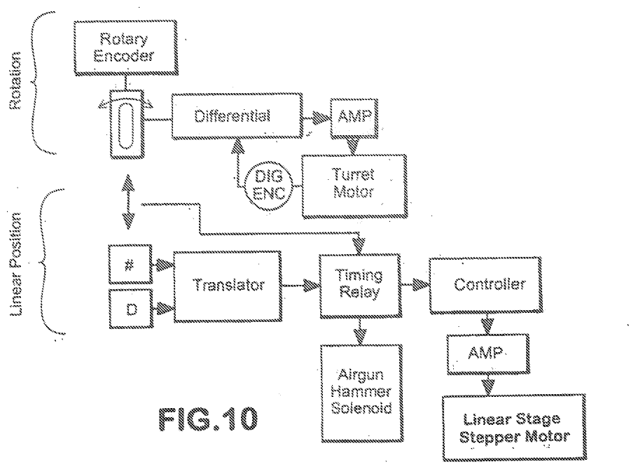

The turret-motor is incapable of significant abrasive action in its rotatory mode, so that in situations where the barrel-assembly is readily rotated by hand, this mode is not needed in the barrel-assembly as independent from the airgun. Since reciprocal transluminal action is manual as well, no joystick control as shown in FIG. 9 and functionally depicted in FIG. 10 is needed. Instead, as shown in FIG. 6, the onboard angioplasty control panel motor control rocker switch marked ‘T-motor’ in the upper left-hand corner switches between the heating and oscillatory modes. Any different oscillatory modes programmed are cycled through by re-depressing the right-hand side of the rocker switch and thus not seen in the control panel shown in FIG. 6.

As shown in FIG. 9, rather than to rotate the barrel-catheter or airgun barrel separately, the mounting used to allow the barrel-catheter about the long axis through the airgun barrel preferably consists of inverted U-shaped cradle swing or swivel bracket 149 bent into heavy gauge strip steel stock by a brake. The vertical side to side connecting segment or bridge portion of the bracket is screwed down to the upper surface of the linear positioning table 150.

Airgun 151 rests on and can be locked at any rotational angle coaxial to the long axis of airgun barrel 152 in compression or tightenable swing cradle bracket 149. The cradle allows adjustability in the angle of rotation in the same way as the elevation adjusting device of a spotlight, except that the spotlight has tightening knobs at both sides while swing cradle bracket 149 has only one tightening knob 154 at the rear. The front component of the airgun enclosure-divided shaft that allows long axis airgun barrel 152 coaxial rotation of airgun 151 consists of airgun barrel 152 itself, while the rear portion consists of male threaded short stud 153 with upward directed pointer, resistance or spot-welded to the back of airgun cabinet 151.

A round scale with the rotational angle marked off in degrees is affixed by means of an adhesive to the rear surface of the rear arm of swing cradle bracket 149 in surrounding relation to the stud hole. Airgun barrel 152 and stud 153 fit through airgun barrel long axis-centered holes toward the upper ends of the front and rear arms of cradle-bracket 149. Rear stud 153 having been passed through the hole in the rear arm of cradle bracket 149, a Belleville disk ring spring washer is placed over the stud, centered in the angle scale and flush against the rear side of cradle bracket 149. Rotating airgun 151 thus rotates the pointer mounted on stud 153 over the scale, indicating the angle of rotation of airgun 151. Tightening knurled knob threaded over stud 153 compresses together the arms of cradle bracket 149 against the front and back of airgun 151 enclosure, fixing the airgun in rotational angle.

When, as shown in FIG. 9, spaces separate the knurled knob at the back and the front of airgun 151 enclosure from the swing cradle bracket, tube spacers (spacer sleeves, spacer tubes) are used to take up the intervals. The knob is tightened so that the rotational angle of the airgun, which is stabilized in angle of rotation by friction, can be adjusted by hand. At the front of the airgun cabinet, the barrel passes through a hole that journals by friction fit a ball bearing that holds the airgun barrel in surrounding relation. The axis of rotation for this airgun swing-type carriage mounting is thus coaxial with the airgun barrel and therefore allows adjustment in the working arc.

Turning now to FIGS. 9 and 10 here and FIG. 85 in the parent application, both the closed-loop control of the turret-motor and open-loop control of the linear stage are initiated by the operator with the joystick, forward to move the table forward, backward to move it backward, and clockwise or counterclockwise to move the turret motor to the corresponding angle. Move and discharge operation is limited to the linear stage or transluminal positioner.

Transluminal runs consisting of translation by the linear stage, holding fast while the timing relay signals the airgun hammer direct current powered plunger solenoid to operate, then executes the following increment, are performed one at a time, direct observation and action cancellation or override by the operator taking precedence over any automatic function. The components shown in FIG. 10, a differential, or comparator, digital encoder, timing relay, amplifier, or operational amplifier, and so on, are universally used in motional control systems and therefore immediately recognized by those familiar with motional control systems.

There is, therefore, no stack or register to store successive transluminal discharge runs, and no provision for the programming of successive runs is allowed. When it is desired to induce oscillation in the translatory or transluminal axis, the linear stage is controlled in a closed loop that may be intentionally derivative gain overdriven or overly amplified. Unless made to progress at a very slow rate, continuous positional control, whether by direct analogy whereby the muzzle-head would be made to move say, one millimeter for each move at the control of a centimeter, or by continuous directional incrementing, so that the muzzle-head would continue to increment in the direction of the control until the control was retracted, are both subject to constant overshooting.

The form of control must not permit a condition such that every change in position requires to be corrected, much less several times. Wasted motion would soon fatigue, prompting sloppiness where this must not be tolerated. While the first of these forms of control is the most intuitive or consistent with spontaneous eye-hand coordination, and the second is more intuitive than control that is based upon strict adherence to a previous setting of controls to specify the number, size, and direction of the increments to be executed automatically, for interventional application, where losses in efficiency based upon essential design factors are unacceptable and impatience with constant overshooting might induce carelessness, the first of these forms of control is rejected and the second reserved for quickly positioning the muzzle-head at the starting position for automatic discharge.

Once initiated, however, the system requires that the number and size of the increments to comprise each movement be entered first and the joystick or cyclic used to indicate the direction of movement, the latter being singular in any one such discharge-run or compound action. The apparatus then automatically switches between the movers (turret-motor and linear stage) and the airgun direct current powered plunger solenoid used to strike the valve body pin, stopping long enough between increments to allow the implants to travel to the trajectory termini. Shifting the joystick forward moves the linear stage stepper motor distad, backward proximad, tilting to the right or rotating clockwise moves the turret-motor clockwise, and tilting to the left or rotating counterclockwise moves the turret-motor counterclockwise.

The joystick has a central null position through which changes from forward (distad) to backward (proximad) direction of the airgun mounting linear positioning table must pass, so that reversal cannot be immediate. Similarly, rotation of the turret-motor cannot be reversed immediately, because a null region separates clockwise from counterclockwise contact, and since forward-backward excursion passes through the rotatory null region, simultaneous actuation of the turret-motor and linear stage is impossible. Actuating the automatic discharge (autodischarge) rocker switch shown in FIG. 85 causes the time delay relay shown in FIG. 84 to alternately switch between either the linear stage stepper or turret-motor as mover to the airgun solenoid that when energized strikes the valve-body pin releasing CO₂ into the airgun chamber causing the implants to be ejected.

The airgun is mounted on a linear positioning table that by moving the airgun bodily, transluminally advances or retracts (withdraws) the muzzle-head. The linear positioning table can be used to a. Accurately reposition the muzzle-head once the barrel-assembly has been inserted into the airgun barrel, which involves only control over the linear stage and turret-motor as movers, b. Reposition the muzzle-head and then effect discharge semiautomatically, the operator manually triggering each discharge, which alternates between positional control and discharge, or c. Direct automatic repositioning and discharge, in which compound action the muzzle-head is manually directed to reposition by uniform distances (increments, stretches) stop at each conjunction by a fixed time that is long enough for the airgun to discharge with the longest barrel-assembly, and then discharge automatically at each stop, which requires the automatic and coordinated control of the movers and the airgun.

Turning now to the airgun control panel shown in FIG. 85 of the parent application, once angioplasty has been completed, the barrel-assembly is inserted into the airgun. The power supply is activated by pressing the ON-OFF toggle switch to the ‘ON’ button. To bring the muzzle-head to the starting position for discharge, the joystick is held in the direction required until the linear stage and the turret-motor have incremented toward and positioned it thus. Semiautomatic discharge is appropriate for isolated discharge, but implantation for stenting demands a proximity and accuracy of adjacent placement that only machine controlled automatic discharge allows to be attained.

Once the starting position has been reached, the airgun can be a. Discharged manually or semiautomatically by releasing the safety on the dead-man trigger switch and depressing the trigger, or b. Semiautomatic discharge initiated by using the upper dial to set the number of increments and the lower dial to set the length in millimeters of each increment. To measure and render observable the extent of linear travel of the linear positioning table, horizontal joint between the base and moving platform of the linear positioning table 150 is calibrated in millimeters. A failure to discharge will be evidement and thus can be distinguished externally, as discussed in the section of the parent application entitled Modes of Failure.

Less desirably, the airgun discharge components proper—CO₂ or compressed air cylinder, valve body, chamber, and barrel—can be separately mounted within the airgun cabinet for rotation on a U-bracket mounted on a linear positioning table, which then contained within the cabinet at the bottom, even when made of transparent polycarbonate plastic with a hinged or removable top that may be left open to allow access to allow adjustment to the valve body slide as described below, is then more likely to obscured from view by reflection.

Such an arrangement thus reduces the observable action of the linear positioning table shown in FIG. 9, of which the incremental moves, at both airgun barrel cabinet portal and entry into the body, are minute and not readily observable. Since this would make a malfunction less noticeable, it is not preferred. Made for a particular barrel-assembly rather than universal, or meant for use with any barrel-assembly, the ablation or ablation or angioplasty control panel shown in FIG. 6 provides controls for setting the current to the turret-motor stator when used as a thermal angioplasty heating element, and either of two electrical radial projection unit thermal expansion wires used to raise brush-type abrasion angioplasty tool-inserts into working position.

The run-ahead downstream embolic or trap-filter, part number 173 in FIG. 3 is simultaneously deployed with any tool-insert such as a side sweeper-scraper that generates debris. FIG. 7 provides a diagrammatic representation of the control components and connections within the power and control housing of a combination-form ablation or ablation and angioplasty-capable barrel-assembly such as shown in FIGS. 71 and 78 of the parent application, wherein each function is assigned to a separate rather than joint microcontroller, the onboard control panel shown here in FIG. 6 corresponding to FIG. 79 in the parent application as referred to in the upper left hand corner of FIG. 7.

1. An extension for the barrel of a variable discharge-pressure medical interventional airgun for use as a hand tool for the insertion into tissue and the introduction into, passage through, and discharge within the lumen of a tubular anatomical structure of medicinal and magnetically susceptible spherules into the wall surrounding said tubular anatomical structure as a means for the delivery of interventional therapy and an aid to imaging.

2. An extension for the muzzle, that is the distal terminus from which the spherules are discharged, from a variable discharge pressure operated airgun as defined in claim 1 wherein at least one tractive electromagnet is mounted proximal thereto so that energizing said electromagnet allows a misplaced spherule to be recovered.

3. An extension for the barrel of a variable discharge-pressure medical interventional airgun for use as a hand tool as defined in claim 1 with an unobstructed passageway entirely through its longitudinal central axis to allow different miniature cabled devices such as scopes, lasers, intravascular ultrasound probes, a rod to deploy an embolic filter at the distal end of said extension, and a rod with a pressure sensor which the operator can view to test the force of expulsion needed to insert the spherule implant into that tissue, each such longitudinally configured device insertable interchangeably during an interventional procedure thereby imparting considerable imaging and therapeutic capability.

4. An extension for the barrel of a variable discharge pressure operated airgun as set forth in claim 1 wherein the current conducted through the coil of said tractive electromagnet can be increased causing the temperature of said coil to be increased to a level suitable for thermal angioplasty and ablation of atheromatous, infected, and malignant tissue.

5. An apparatus for stenting a tubular anatomical structure whereby a variable discharge pressure medical interventional airgun as set forth in claim 1—is used to implant ferromagnetic spherules within the tissue lining the lumen of said tubular structure, said spherules susceptible to a magnetically generated tractive force in relation to magnetized material about the external surface of said tubular anatomical structure.

6. An extension for the barrel of a variable discharge-pressure medical interventional airgun used as a hand tool for the insertion into tissue of small therapeutic, magnetically susceptible, and radiopaque spherules.

7. A medical interventional airgun incorporating a rotary spherule airgun loading magazine clip whereby each in a plurality of spherules is released into a different discharge tube for simultaneous expulsion and insertion at a different radial angle into the surrounding wall of a tubular anatomical structure.

8. A medical interventional airgun barrel incorporating radially outward thermal expansion wire-projectable lift platforms which in order to medically treat a tissue surface, lift interchangeable tool inserts into working contact with said tissue surface such as that of the wall surrounding a lumen obstructed by a deposition of crystal or an atheromatous lesion.

9. A therapeutic spherule containing magnetically susceptible matter exclusively matched to and limited to implantation by a corresponding dedicated medical interventional airgun such that said airgun and said spherule stand in a reciprocal relation as to define a unit invention.
